(19) United States
(12) Patent Application Publication
(10) Pub. No.: US 2017/0199178 A1
(43) Pub. Date: Jul. 13, 2017

(54) SOLID PHASE LABELING METHOD

(71) Applicant: MU Bioteknik AB, Stockholm (SE)

(72) Inventors: Mathias UHLÉN, Stocksund (SE); Hélène ANDERSSON SVAHN, Stockholm (SE); Emma LUNDBERG, Stockholm (SE)

(73) Assignee: MU Bioteknik AB, Stockholm (SE)

(21) Appl. No.: 15/471,677

(22) Filed: Mar. 28, 2017

Related U.S. Application Data
(63) Continuation of application No. 14/843,661, filed on Sep. 2, 2015.
Application No. 14/843,661 is a continuation of application No. 12/076,661, filed on Mar. 20, 2008, now abandoned.
(60) Provisional application No. 60/907,190, filed on Mar. 23, 2007.

(51) Int. Cl. G01N 33/533; G01N 33/545; G01N 33/548

(52) U.S. Cl. CPC G01N 33/533; G01N 33/548; G01N 33/545

(57) ABSTRACT

The invention provides for the labeling of antibodies with a fluorescent label, using a solid support comprising an affinity for an Fc portion of an antibody. Thus, the invention provides a method for labeling an antibody or fragment thereof with a fluorescent label, comprising the steps of immobilizing an antibody on the solid support and covalently coupling a fluorescent label to the immobilized antibody, as well as a kit for performing such method.

### CROSS REFERENCE TO RELATED APPLICATIONS

This application is a Continuation Application of co-pending U.S. application Ser. No.: 14/843,661 filed Sep. 2, 2015, which is a Continuation Application of co-pending U.S. application Ser. No. 12/076,661 filed on Mar. 20, 2008, now abandoned, which claims priority under 35 U.S.C. 119(e) to U.S. Provisional Application No. 60/907,190 filed on Mar. 23, 2007, all of which are hereby expressly incorporated by reference into the present application.

### FIELD OF THE INVENTION

The present invention relates to the field of labeling of proteins, in particular antibodies.

### BACKGROUND OF THE INVENTION

Many applications in cell biology require use of antibodies for specific interactions and labeling of these antibodies with detector molecules for visualization. These detector molecules can, dependent on the application, be bioluminescent markers, enzymes or fluorescent labels. Fluorescently labeled antibodies are a very important tool in this field to provide for the specific detection of antigens. Fluorescently labeled antibodies are commonly used in immunohistochemistry, fluorescent microscopy, protein arrays, flow cytometry, Western blots etc, and are the reagents of choice for quantification of antigens in all of these applications. There are mainly two ways to detect antigens with fluorescent labels: either by using a fluorescently labeled primary antibody which binds to the antigen or, alternatively, by using a fluorescently labeled secondary antibody, with affinity for a primary antibody which binds the antigen. An advantage of using labeled primary antibodies is the reduced staining time and the multiplexing possibilities. Labeled primary antibodies allow simultaneous visualization of several antibodies from the same animal species in the same system, for example in the same protein array, cell or tissue, without risking cross-reactivity.

Today, most methods for fluorescent labeling of antibodies are based on labeling in solution. Independently of the coupling chemistry used, a prerequisite of a successful labeling reaction is a pure antibody sample at a high concentration. The labeling reaction in solution is difficult to control and the resulting degree of labeling is hard to predict. The manufacturers of fluorescent dyes therefore often recommend performing several labeling reactions simultaneously with different amounts of fluorescent labels to ensure at least one successful labeling reaction. When the labeling reaction is finished, unreacted fluorescent label must be removed by dialysis or desalting columns adding one additional, time-consuming step. Other disadvantages include dilution and loss of antibody during post-reaction purification steps as well as incomplete removal of free dye.

Further, a few labeling methods using a solid phase have been described. WO97/17610 discloses fluorescent labeling of antibodies bound to antigens immobilized on a solid phase. In this method, the labeling of a specific antibody requires the preparation of a solid phase with the corresponding antigen. Consequently, the method must be specifically adapted to each antibody to be labeled.

Strachan E A et al, J Mol Recognit 17:268-274 (2004), disclose a solid phase biotinylation protocol developed for biotinylation of small amounts (0.1 mg to 10 mg) of antibody on an IMAC support. This protocol is faster than the conventional solution-phase protocols, but still requires a pure antibody sample.

Victoria E J et al, J Immunol Methods 50:205-211 (1982), disclose radiolabeling of antibodies on solid phase protein A. Here, lactoperoxidase-catalyzed iodination, i.e. an enzymatic method, with ¹²⁵I or iodine monochloride (ICl) labeling is utilized. However, radiolabeling on protein A did not prove very effective, as the isotope incorporation was only 18% and 34% for ¹²⁵I and ICl, respectively.

There is a great need for fluorescently labeled primary antibodies, but reproducibility problems, insufficient recovery of antibody and requirement of pure samples at a high concentrations often makes it impossible to obtain such antibodies using existing methods. Efforts have been made to meet some of these needs. However, the need for a protocol applicable for fluorescent labeling of various classes and subclasses of antibodies from a variety of species still remains.

### DISCLOSURE OF THE INVENTION

A novel method for solid phase fluorescent labeling antibodies has been invented. By using a solid support throughout the labeling, excess reagents may be easily washed away while the antibody remains bound to the solid support. The solid support utilized in the inventive method comprises an affinity for an Fc portion of an antibody, e.g. a protein A affinity medium or a protein G affinity medium. Strong and specific interaction between such a solid support and antibodies, or fragments thereof retaining the Fc portion, in contrast to, e.g., the less specific interaction between an IMAC support and antibodies, allows for simultaneous purification, concentration and labeling of antibody samples. Since the solid support may be designed to interact with the Fc portion of various classes and subclasses of antibodies from a variety of species, the method need not be specifically adapted to different specific antibodies. Starting with 100-2000-fold lower antibody concentrations than what is required for labeling in solution, μg amounts of antibodies have been labeled with the inventive method. Further, the method provides a homogeneous labeling environment, which makes it possible to control the degree of labeling by adding different amounts of reactive dye, even if starting with impure antibody samples of low concentration, such as serum.

In a first aspect, the invention relates to a method for labeling an antibody, or fragment thereof retaining the Fc portion, with a fluorescent label, comprising the steps:

immobilizing the antibody or fragment thereof on a solid support comprising an affinity for an Fc portion of the antibody or fragment thereof;

covalently coupling a fluorescent label comprising a reactive group to the immobilized antibody or fragment thereof; and

eluting the labeled antibody or fragment thereof with an eluting buffer.

Throughout the description and the claims, a “solid support” refers to an insoluble, functionalized material to which reagents may be attached, allowing them to be separated from, e.g., excess reagents, contaminants and solvents. Examples of solid supports comprise functionalized polymeric materials, e.g., agarose, or its bead form Sepharose®, dextran, polystyrene and polypropylene, or mixtures thereof; compact discs comprising microfluidic channel structures; protein array chips; membranes, e.g., nitrocellulose or PVDF membranes; microparticles, e.g., paramagnetic or non-paramagnetic beads; pin structures; stick structures, e.g., dip sticks; sensor surfaces for use in surface plasmon resonance instruments; and cell surfaces.

Further, throughout the description and the claims, an “antibody”, or “fragment thereof retaining the Fc portion”, refers to any natural or synthetic protein comprising an Fc portion of an antibody.

Also, throughout the description, “dye” and “label” are used interchangeably to designate fluorescent molecules.

In one embodiment of the first aspect, the fluorescent label is selected from the group consisting of organic fluorophores and fluorescent nanocrystals, such as Quantum dots.

In another embodiment of the first aspect, the solid support is selected from a protein A affinity medium and a protein G affinity medium. Protein A and protein G are bacterial proteins from Staphylococcus and Streptococcus, respectively, that bind with high affinity to the Fc portion of various classes and subclasses of antibodies from many species. A “protein A affinity medium” and a “protein G affinity medium” each refer to a solid phase onto which is bound a natural or synthetic protein comprising an Fc-binding domain of protein A or protein G, respectively, or a mutated variant or fragment of an Fc-binding domain of protein A or protein G, respectively, which variant or fragment retains the affinity for an Fc-portion of an antibody. For example, the solid phase may be a polymeric material such as agarose, or its bead form Sepharose®, dextran, polystyrene and polypropylene, or mixtures thereof.

In another embodiment of the first aspect, the immobilizing comprises the steps:

applying an antibody sample comprising the antibody or fragment thereof to the solid support; and

washing the solid support with a first washing buffer.

As a non-limiting example, the antibody sample is a non-purified solution of the antibody, or fragment thereof retaining the Fc portion. Such non-purified solution may comprise any impurities, such as proteinaceous contaminants, e.g. albumin. For example, the non-purified solution may be diluted or non-diluted serum.

The main purpose of the washing of solid support with the first washing buffer is to remove impurities, such as non-bound proteins, e.g. albumin. The first washing buffer may be any appropriate washing buffer known to the skilled person.

In another embodiment of the first aspect, the covalent coupling comprises the steps:

applying a labeling solution comprising a predetermined concentration of the fluorescent label to the solid support;

incubating the resulting mixture of the labeling solution and the immobilized antibody; and

washing the solid support with a second washing buffer.

As a non-limiting example, the labeling solution comprises a labeling buffer with a pH in the range of 7.3-9.3, e.g. about 8.3.

As another non-limiting example, the predetermined concentration of the fluorescent label corresponds to an amount of the fluorescent label which is 6-10 times (mol/mol) the total amount of antibody or fragment thereof to be labeled and proteinaceous material comprised in the solid support.

The main purpose of the washing of the solid support with the second washing buffer is to remove non-reacted fluorescent label. The second washing buffer may be any appropriate washing buffer known to the skilled person. The first and the second washing buffer may be the same or different.

The eluting buffer may be any appropriate eluting buffer known to the skilled person. For example, at pH 4 or lower, the antibody or fragment thereof dissociates from the solid support comprising an affinity for an Fc portion of an antibody. Thus, in another embodiment of the first aspect, the pH of the eluting buffer is 4 or lower, such as below 4, such as 0.2-3.9, such as 1-3.9, such as 2-3.9, such as 3-3.9, such as 3-3.6.

In another embodiment of the first aspect, the reactive group is selected from an amine reactive group and a sulfhydryl reactive group. Amine reactive groups and sulfhydryl reactive groups bind covalently with lysine residues and cysteine residues, respectively.

As a non-limiting example, the reactive group is an amine reactive group.

As another non-limiting example, the fluorescent label comprising a reactive group is selected from succinimidyl ester of Alexa Fluor® 488 carboxylic acid (mixed isomers), succinimidyl ester of Alexa Fluor® 555 carboxylic acid and succinimidyl ester of Alexa Fluor® 647 carboxylic acid.

Alternatively, the fluorescent label comprising a reactive group is selected from tetrafluorophenyl (TFP) ester of Alexa Fluor® 488 carboxylic acid (mixed isomers), tetrafluorophenyl (TFP) ester of Alexa Fluor® 555 carboxylic acid and tetrafluorophenyl (TFP) ester of Alexa Fluor® 647 carboxylic acid.

In another embodiment of the first aspect, the solid support is arranged inside a solid support container, for example selected from a micropipette tip and a spin column.

As a non-limiting example, the solid support is arranged inside a micropipette tip.

As another non-limiting example, the solid support may be fixated inside the solid support container. Throughout the description and the claims, “fixation” refers to any means for retaining the solid support inside the solid support container during the labeling of the antibody or fragment thereof. For example, the fixation could be such that the solid support is retained inside the container during centrifugation at 400 g.

In another embodiment of the first aspect, the solid support may comprise paramagnetic beads. The use of paramagnetic beads allows for automated labeling applications using magnet-equipped pipetting robot, wherein multiple labeling reactions are performed simultaneously. Thus, as a non-limiting example, one or more of the above-mentioned steps may be performed by a magnet-equipped pipetting robot. For example, the magnet-equipped pipetting robot may be a Magnatrix 8000 Workstation (MBS, Stockholm, Sweden).

In a second aspect, the invention relates to a kit for labeling an antibody, or fragment thereof retaining the Fc portion, comprising:

at least one solid support container containing a solid support comprising an affinity for an Fc portion of an antibody; and

at least one container containing a fluorescent label comprising a reactive group.

The solid support container may be any container suitable for containing a solid support. The kit may be adapted to transferring of the solid support from the solid support container to another container before the labeling of the antibody or fragment thereof is performed. Alternatively, the solid support container is adapted to hold the solid support during the labeling of the antibody or fragment thereof.

As a non-limiting example, if the solid support container is adapted to hold the solid support during the labeling of the antibody or fragment thereof, the solid support may be fixated inside the solid support container.

As another non-limiting example, the solid support container may be selected from a micropipette tip and a spin column.

In another embodiment of the second aspect, the kit further comprises at least one container containing a labeling buffer, and the fluorescent label is in a form adapted to being dissolved in the labeling buffer.

As a non-limiting example, the pH of the labeling buffer is in the range of 7.3-9.3, e.g. about 8.3.

As another non-limiting example, the fluorescent label is in freeze dried form.

In another embodiment of the second aspect, the kit further comprises at least one container containing an eluting buffer for eluting fluorescently labeled antibodies from the solid support. For example, the pH of the eluting buffer may be 4 or lower, such as below 4, such as 0.2-3.9, such as 1-3.9, such as 2-3.9, such as 3-3.9, such as 3-3.6.

In another embodiment of the second aspect, the kit further comprises at least one container containing a washing buffer for washing non-bound material off the solid support. The washing buffer may be any appropriate washing buffer known to the skilled person.

In another embodiment of the second aspect, the solid support is selected from a protein A affinity medium and a protein G affinity medium as defined in the discussion about the first aspect above.

In another embodiment of the second aspect, the reactive group is selected from an amine reactive group and a sulfhydryl reactive group. Amine reactive groups and sulfhydryl reactive groups bind covalently with lysine residues and sulfhydryl groups respectively.

As a non-limiting example, the reactive group is an amine reactive group.

As another non-limiting example, the fluorescent label comprising a reactive group is selected from succinimidyl ester of Alexa Fluor® 488 carboxylic acid (mixed isomers), succinimidyl ester of Alexa Fluor® 555 carboxylic acid and succinimidyl ester of Alexa Fluor® 647 carboxylic acid.

### BRIEF DESCRIPTION OF THE FIGURES

FIG. 1 shows the amount of eluted antibody in each 50 μl fraction eluted from a protein A micropipette tip. The antibody was eluted using 0.2 M HAc, pH 3.3. The x-axis corresponds to the 8 different fractions where 1 is the first fraction and 8 is the last. The y-axis corresponds to the amount of eluted antibody on a μg scale.

FIG. 2 shows the degree of labeling obtained after conjugating Alexa Fluor® 555 to rabbit-α-His₆-ABP on protein A affinity medium in different labeling buffers at different pH. The x-axis shows the pH of the different labeling buffers. The y-axis corresponds to the degree of labeling obtained in terms of mole Alexa Fluor® 555 dye per mole antibody.

FIG. 3 shows the degree of labeling obtained after conjugating Alexa Fluor® 555 to rabbit-α-His₆-ABP on solid phase on protein A affinity medium with different incubation times for the conjugating reaction. The x-axis shows the incubation time and temperature during labeling. The y-axis corresponds to the degree of labeling obtained in terms of mole Alexa Fluor® 555 dye per mole antibody.

FIG. 4 shows the degree of labeling obtained after conjugating Alexa Fluor® 555 to 14 μg rabbit-α-His₆-ABP on protein A affinity medium varying with the different amounts of added dye. The x-axis shows the amount of added Alexa Fluor® 555 dye in nmol. The y-axis corresponds to the degree of labeling obtained in terms of mole Alexa Fluor® 555 dye per mole antibody.

### DETAILED DESCRIPTION OF AN EXAMPLE EMBODIMENT

### Materials and Methods

### Materials

All chemicals were purchased from Sigma-Aldrich (Stockholm, Sweden) unless otherwise noted. The following Alexa Fluor® dyes were supplied by Invitrogen-Molecular Probes (Eugene, Oreg., US): succinimidyl ester of Alexa Fluor® 488 carboxylic acid (mixed isomers), succinimidyl ester of Alexa Fluor® 555 carboxylic acid and succinimidyl ester of Alexa Fluor® 647 carboxylic acid. 1 mg of each type of Alexa Fluor® dye was dissolved in DMSO to a final concentration of 40 μg/μl and stored at −80 ° C. Mono-specific polyclonal rabbit antibodies were obtained from the Human Protein Atlas program (www.proteinatlas.org). Antibodies used were α-His₆-ABP (ABP refers to albumin binding protein), α-C1 tetrahydrofolate synthase, α-ornithine carbamoyltransferase, α-programmed cell death 4 isoform 1 and α-activator of 90 kDa heat shock protein ATPase homolog 1 (AHA1). Alexa Fluor® 555 rabbit anti-mouse IgG were obtained from Invitrogen-Molecular Probes (Eugene, Oreg., US). Slide-A-Lyzer dialysis cassettes were obtained from Pierce Biotechnology (Rockford, Ill., US) and NAP-5 desalting columns from GE Healthcare (Uppsala, Sweden). Micropipette tips (TopTip™ 10-200 pl) filled with protein A affinity medium (protein A bound to agarose) were obtained from Glygen Corp (Colombia, Md., US).

Covalent Labeling of Antibodies with Alexa Fluor® Dyes in Solution

All procedures with Alexa Fluor® dyes were carried out in dim light. Rabbit α-His₆-ABP IgG was purified using Protein A Sepharose Fast Flow (GE Healthcare, Uppsala, Sweden) to remove albumin before being labeled with Alexa Fluor® 555 in solution according to the manufacturer's instructions. To determine a proper pH for labeling, 10 μl of 1 M NaHCO₃ buffer, pH 8.3, was added to 90 μl of antibody diluted in PBS, giving a final antibody concentration of 6 mg/ml. A 10-fold molar excess of Alexa Fluor® 555 dye was added to the antibody sample and the conjugation was allowed to proceed for 1 hour at room temperature before free dye was removed with a NAP-5 desalting column. To ensure that all free dye was properly removed, this was repeated with a new NAP-5 column. The antibody and Alexa Fluor® Dye concentrations were measured as described below.

Covalent Labeling of Antibodies with Alexa Fluor® Dyes on Solid Phase

All procedures with Alexa Fluor® dyes were carried out in dim light. Protein A micropipette tips (TopTip™ tips) were used as mini-spin columns with centrifuge-adaptors holding them in place in eppendorf tubes according to the manufacturer's (Glygen's) instructions. To force liquid through the micropipette tips, centrifugation at 400 g for 10-60 seconds depending on volume was performed.

Protein A micropipette tips were conditioned with 100 μl washing buffer (15 mM NaH₂PO₄, 500 mM NaCl, pH 8.0) before 5-20 μg of antibody was added and mixed with the protein A matrix. The antibody was allowed to bind for one minute before the liquid was forced through the column and the procedure was repeated 2-5 times for maximal binding. Albumin and other non-IgG proteins were washed out twice with 150 μl washing buffer before equilibration with 100 μl labeling buffer. Alexa Fluor® dye from the DMSO stock was mixed with labeling buffer to a final volume of 15 μl. Unless otherwise noted, 8 nmol of dye was used. The labeling buffer was 100 mM NaCl, 35 mM H₃BO₃, pH 8.3, unless otherwise noted. The dye mixture was added to the micropipette tips and mixed with the protein A matrix-antibody complexes and conjugation was allowed to proceed for 1-3 hours at room temperature or at 4° C. over night. It was noted that if more than 5-7% (v/v) DMSO was added to the antibody-protein A-buffer mixture, the labeling reaction was inhibited. Unreacted dye was removed by five washings with washing buffer. The Alexa Fluor® dye labeled antibody was eluted with 50-500 μl of eluting buffer depending on desired antibody concentration. The eluting buffer was 0.2 M HAc, pH 3.3. The pH of the eluate was raised immediately after the elution by addition of ¼ of the elution volume of 0.5 M Tris, pH 8.5. Dye and antibody concentrations were measured before the addition of BSA to a final concentration of 0.1% (w/v) for stabilization of the antibodies. The buffer was changed to PBS with either dialysis or dilution.

### Measurement of Antibody and Dye Concentrations

The Alexa Fluor® dye concentrations were determined from the visible light absorption spectrum, according to the manufacturer's instructions, using published extinction coefficients (E₄₉₆=71000 M⁻¹cm⁻¹ for Alexa Fluor® 488, ε₅₅₅=150000 M⁻¹cm⁻¹ for Alexa Fluor® 555, ε₆₅₀=239000 M⁻¹cm⁻¹ for Alexa Fluor® 647). The concentration of labeled antibodies was determined from an UV-VIS absorption spectrum (E₂₈₀=170000 M⁻¹cm⁻¹) taking into account the absorbance contribution from the covalently bound Alexa Fluor® Dye at 280 nm (CF₂₈₀=0.11 for Alexa Fluor® 488, CF₂₈₀=0.08 for Alexa Fluor® 555, CF₂₈₀=0.03 for Alexa Fluor® 647) according to the manufacturer's instructions. All absorbance measurements were performed with a Cary 50 UV-Visible Spectrophotometer (Varian, Palo Alto, Calif., US).

### Fluorescence Measurements of Free Alexa Fluor® Dyes and Alexa Fluor®-Conjugated Antibodies

Unconjugated Alexa Fluor® dyes 488, 555 and 647 were diluted to a final concentration of 1 μM in the following buffers: PBS pH 7.2, 0.1 M sodium citrate pH 2.5, 0.1 M sodium citrate pH 3.0, 0.1 M sodium citrate pH 3.5, 0.1 M sodium citrate pH 4.0, 0.1 M sodium citrate pH 4.5 and 0.2 M acetic acid pH 3.3; and incubated for one hour before pH was raised by the addition of ¼ volume of Tris pH 8.5, following which the fluorescence was read.

A rabbit-α-His6-ABP antibody labeled with Alexa Fluor® 555 using the solid phase protocol described herein, with a resulting average incorporation of 2.4 mole dyes per mole antibody, and a commercial rabbit-α-mouse IgG conjugated with Alexa Fluor® 555 were diluted to a final concentration of 0.1 μM in PBS before fluorescence was read.

All fluorescence measurements were performed at 25° C. using a Perkin Elmer Luminescence Spectrometer LS50B (Waltham, Mass., US). The dyes were excited at λₘₐₓ (λₘₐₓ=495 for Alexa Fluor® 488, λₘₐₓ=555 for Alexa Fluor® 555, λₘₐₓ=650 for Alexa Fluor® 647) and the fluorescence was read at the emission maxima (Emₘₐₓ=519 for Alexa Fluor® 488, Emₘₐₓ=565 for Alexa Fluor® 555, Emₘₐₓ=665 for Alexa Fluor® 647), all according to the manufacturer's instructions.

### Results

### Effect of Low pH on Alexa Fluor® Dye Fluorescence

According to the manufacturer, the Alexa Fluor® dyes are stable between pH 4-10 (Haugland R, (2005) “The handbook—A Guide to fluorescent probes and labeling technologies”, M.T.Z Spence ed. (Eugene, OR: Invitrogen-Molecular Probes)). To be able to effectively elute the labeled antibodies from a protein A matrix, a lower pH around 3 is typically used. To test whether the Alexa Fluor® dyes could withstand a prolonged exposure in a low pH environment, Alexa Fluor® 488, Alexa Fluor® 555 and Alexa Fluor® 647 were diluted in different buffers with pH ranging from 2.5 to 7, to a final concentration of 1 μM, and incubated for one hour before the pH was raised by the addition of ¼ volume of Tris. The fluorescence was read and there were no significant differences between the fluorescence of any of the fluorescent labels in the different buffers compared to that in PBS. This shows that elution of labeled antibodies with 0.2 M acetic acid pH 3.3 does not lower the fluorescence of the conjugated dye and can thus be used for efficient elution of labeled antibody from the protein A affinity media.

### Elution of Antibody

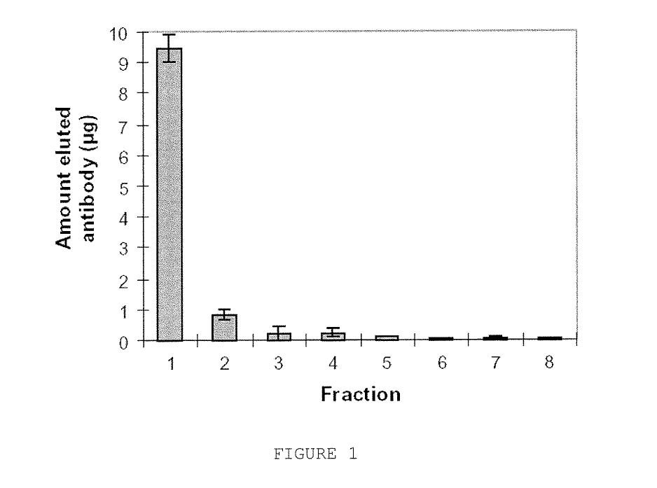

To evaluate the volume needed for efficient elution of labeled antibody from micropipette tips containing protein A affinity media, 14 μg of antibody was allowed to bind to the protein A before albumin was washed away and the antibodies were eluted in 50 μl fractions. 87% of the eluted antibodies were collected in the first fraction (FIG. 1). This shows that it is possible to elute the labeled antibody in a small volume and obtain a more concentrated sample than the starting sample. The elution volume can be further lowered by passing the eluate several times through the micropipette tip.

### Effect of pH of Labeling Buffer During Solid Phase Labeling

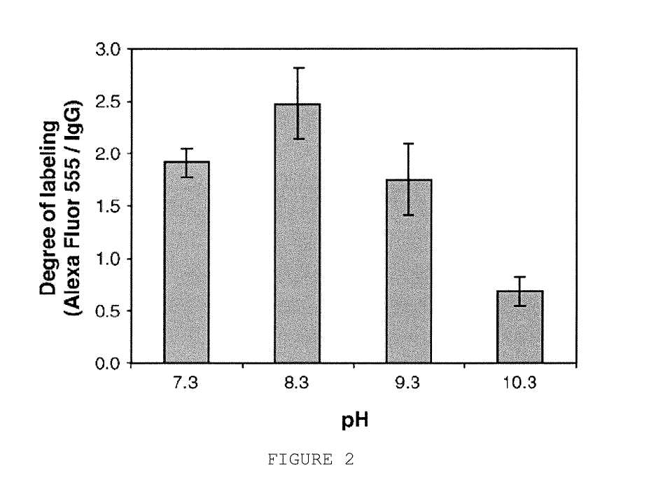

To evaluate how the pH of the labeling buffer effects the conjugation of fluorescent dye to antibodies, 14 μg of rabbit α-His₆-ABP IgG was labeled with Alexa Fluor® 555 on protein A in micropipette tips in four different labeling buffers with different pH. The buffers were: a) 50 mM phosphate, pH 7.3; b) 100 mM NaCl, 35 mM H₃80₃, pH 8.3; c) 100 mM Na₂CO₃, pH 9.3 and d) 100 mM Glycin-NaOH, pH 9.3. Results show that for a 2 hour incubation time, labeling using the buffer containing 100 mM NaCl and 35 mM H₃BO₃ at pH 8.3 was superior to the other buffers, giving an average of 2.5 incorporated fluorescent labels per antibody molecule in comparison to a degree of labeling lower than two fluorescent labels per antibody for the other buffers (FIG. 2). This correlates well with the manufacturer's recommendations of using 0.1-0.2 M NaHCO₃, pH 8.3, for conjugation with succinimidyl esters. All subsequent solid phase labelings were performed with a labeling buffer containing 100 mM NaCl and 35 mM H₃BO₃ at pH 8.3.

Effect of Labeling Time And Temperature

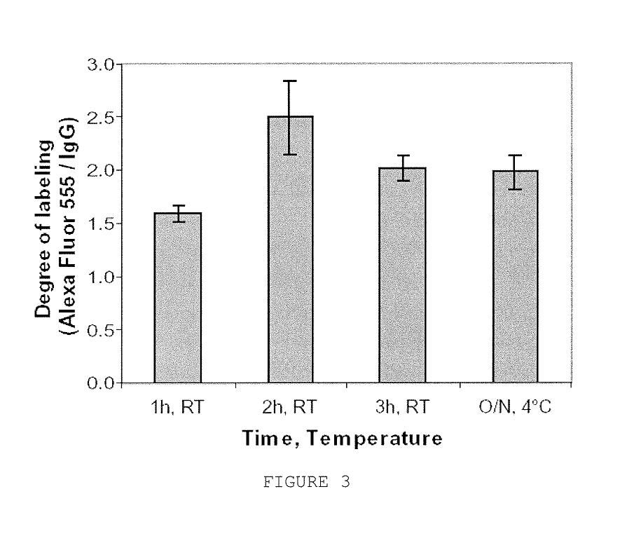

The next step was evaluation of the incubation time and temperature resulting in the maximal number of incorporated fluorescent labels per antibody. Rabbit α-His₆-ABP IgG was labeled with Alexa Fluor® 555 using the solid phase labeling protocol described above and the reactions were allowed to proceed for 1, 2 or 3 hours at room temperature or over night at 4° C. The results show that on average 1.6 fluorescent labels had been incorporated per molecule antibody after one hour, and that the number was raised to 2.4 after 2 hours (FIG. 3). Proceeding with the labeling for 3 hours at room temperature did not give a higher degree of labeling. This indicates that 1 hour of incubation, under the certain conditions used in this example, may not be sufficient for obtaining a complete conjugating reaction, while 2 hours seemed to be sufficient for completing the reaction. However, a shorter incubation time may be compensated by a higher fluorophore concentration. Labeling overnight at 4° C. gives a degree of labeling comparable to that after 2 hours at room temperature, and may be a good option when labeling unstable or sensitive antibodies. In all further solid phase labelings, the conjugation was allowed to proceed for 2 hours at room temperature.

Effect of Amount of Dye for Labeling

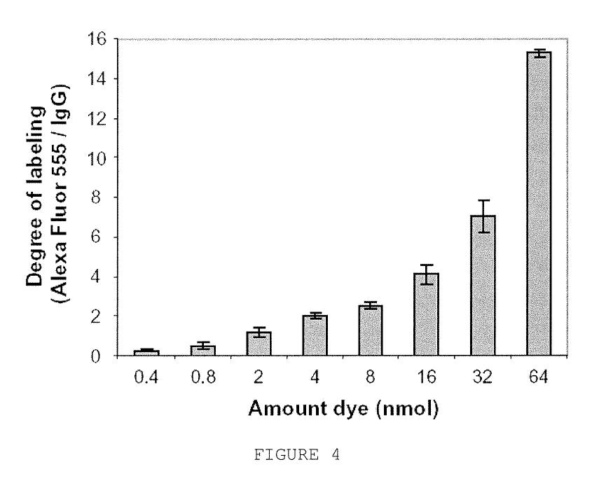

To evaluate the effect of dye concentration, 14 μg of rabbit α-His₆-ABP IgG was labeled in protein A micropipette tips with different amounts of Alexa Fluor® 555 dye. The results are presented in FIG. 4. When labeling on solid phase, both protein A on the affinity medium and the bound antibody will be labeled. Therefore, the molar excess of dye must be compared to the molar amount of antibody plus protein A. According to the manufacturer, one micropipette tip contains about 30-50 μg protein A. This taken together with 14 μg of antibody implies a total amount of protein of approximately 0.8-1.3 nmol. Use of 0.4 nmol of Alexa Fluor® 555 resulted in a degree of labeling (DOL=the number of moles of dye incorporated per mole of antibody) of approximately 0.26, and using 64 nmol of the dye resulted in a DOL of 15. Consequently, it is clear that adding more dye results in a higher degree of labeling. The different DOL-values could be reproduced several times, showing the robustness of the method. These results show that predictable degrees of labeling can easily and reproducibly be achieved by adjusting the amount of added fluorescent label, using the solid phase method according to the invention. For some applications, one incorporated fluorescent label is optimal while in other cases, a maximal DOL may be desirable. To achieve a reproducible DOL, which is high enough to produce bright signals yet low enough to avoid interference with antigen binding activity, and to minimize the amount of used dye, the addition of 8 nmol dye, corresponding to approximately 6-10 times molar excess, was chosen for all further solid phase labelings.

### Fluorescence of Solid Phase Fluorescently Labeled Antibodies

The fluorescence of the antibodies labeled using the solid phase labeling protocol according to the invention was evaluated by comparing such antibodies to the same type of antibodies labeled with the soluble protocol recommended by the manufacturer and to a commercial Alexa Fluor® 555 conjugated antibody. Rabbit α-His₆-ABP IgG was labeled in solution with Alexa Fluor® 555 according to the manufacturer's instructions as follows: albumin was removed using a protein A affinity column, and a 10-fold molar excess of dye was used to label the antibody at a concentration of 6 mg/ml of antibody. This resulted in an average of 2.5 incorporated fluorescent labels per antibody, which corresponds well to the DOL that is obtained using the solid phase labeling protocol according to the invention with a 6-10-fold molar excess of dye. Antibodies labeled with Alexa Fluor® 555 in solution and on solid phase as well as a commercial Alexa Fluor® 555-rabbit-anti-chicken IgG, were diluted to 17 μg/ml in PBS before measurement of fluorescence. There were no significant differences in the fluorescence intensity between the three antibodies. These results show that the two labeling methods were comparable in terms of labeling efficiency, given a certain molar excess of dye, and that the resulting labeled antibodies showed a fluorescence intensity comparable to that of a commercial fluorescently labeled antibody.

### Reproducibility of the Solid Phase Labeling Protocol According to the Invention

To evaluate the reproducibility of the solid phase labeling method according to the invention, 8 antibody samples were labeled. Four of the antibody samples were labeled with Alexa Fluor® 555 and the other four with Alexa Fluor® 647 using the following solid phase labeling protocol: 10 μg of each antibody sample were diluted with the labeling buffer (pH 8.3, 100 mM NaCl, 35 mM H₃BO₃) to concentrations ranging from 0.02-0.15 mg/ml, and mixed with 8 nmol of dye (approximately 6-10 times molar excess) and incubated for 2 hours at room temperature. The labeling with Alexa Fluor® 555 generated an average DOL of 2.1±0.08 while labeling with Alexa Fluor® 647 generated an average DOL of 4.1±0.4. This shows that this labeling method is applicable for labeling different antibodies at different concentrations straight from a mixture of antibody and albumin in less than 3 hours and that it will still generate a reproducible result with a precise and predictable DOL. The fact that the average DOL obtained when labeling with Alexa Fluor® 647 is higher than when labeling with Alexa Fluor® 555 shows that the amount of dye needed to generate antibodies with a specific DOL might vary between different fluorescent labels and have to be adjusted for each fluorescent label.

Labeling of IgG from Rabbit Serum

IgG from rabbit sera was labelled with Alexa Fluor® 647 on solid phase using the solid phase labeling protocol described above. The labelling experiments were performed in triplicates. 30 μ rabbit sera (HPA014840) was loaded in three separate tips, assumably saturating the protein A present in the micropipette tip. The amount of reactive Alexa Fluor® 647 dye added to each micropipette tip was 16 nmoles. The labeling reaction was allowed to proceed for 2 hours at room temperature. The labelled antibody was eluted with 50 μl elution buffer (0.2 M HAc pH 3.3) and the pH was raised by adding 22.5 μl 1 M Tris buffer pH 11.

The measurements of degree of labeling was performed according to previously described protocol and resulted in an average degree of labeling of 1.9 fluorophores per antibody molecule, with a standard deviation of 0.10. The labeled rabbit IgG was also verified on a SDS-Page gel, showing a pure sample with bands with molecular weight corresponding to the heavy and light chain of rabbbit IgG.

This shows that the disclosed solid phase labeling method is efficient when labeling antibodies of low concentration in a complex media, such as serum. The high reproducibility found when labeling in serum also verifies the high robustness and reproducibility of the disclosed method.

1. A method for labeling a natural or synthetic protein comprising an Fc portion of an antibody with a fluorescent label, comprising the steps:
immobilizing the natural or synthetic protein comprising an Fc portion of an antibody on a solid support comprising a protein A affinity medium or a protein G affinity medium, wherein the solid support is arranged inside a micropipette tip; said immobilizing step comprising:
applying a non-purified solution of a sample comprising 5-20 μg of the natural or synthetic protein to the solid support;
washing the solid support with a first washing buffer;
covalently coupling a fluorescent label comprising a reactive group to the immobilized natural or synthetic protein, said coupling step comprising:
applying a labeling solution to the solid support, wherein the labeling solution comprises the fluorescent label at a concentration of 6-10 times molar excess of the total amount of natural or synthetic protein to be labeled and proteinaceous material comprised in the solid support;
incubating the resulting mixture of the labeling solution and the immobilized natural or synthetic protein; and
washing the solid support with a second washing buffer; and
eluting the labeled natural or synthetic protein with an eluting buffer.

2. A method for labeling an antibody, or fragment thereof retaining an Fc portion of the antibody, with a fluorescent label, comprising the steps:
immobilizing the antibody or fragment thereof on a solid support comprising a protein A affinity medium or a protein G affinity medium, wherein the solid support is arranged inside a micropipette tip, said immobilizing step comprising:
applying a non-purified solution of a sample comprising 5-20 μg of the antibody or fragment thereof to the solid support; and
washing the solid support with a first washing buffer;
covalently coupling a fluorescent label comprising a reactive group to the immobilized antibody or fragment thereof, said coupling step comprising:
applying a labeling solution to the solid support, wherein the labeling solution comprises the fluorescent label at a concentration of 6-10 times molar excess of the total amount of antibody or fragment thereof to be labeled and proteinaceous material comprised in the solid support;
incubating the resulting mixture of the labeling solution and the immobilized antibody or fragment thereof; and
washing the solid support with a second washing buffer; and
eluting the labeled antibody or fragment thereof with an eluting buffer.

3. The method according to claim 2, wherein the antibody sample comprises serum.

4. The method according to claim 2, wherein the eluting buffer has a pH of 4 or lower.

5. The method according to claim 2, wherein the reactive group is selected from the group consisting of an amine reactive group and a sulfhydryl reactive group.

6. The method according to claim 5, wherein the reactive group is an amine reactive group.

7. The method according to claim 2, wherein one or more steps is performed by a magnet-equipped pipetting robot.

8. The method according to claim 2, wherein the fluorescent label comprises an organic fluorophore.

9. The method according to claim 2, wherein the antibody is a complete antibody.

10. A kit for labeling an antibody or fragment thereof retaining an Fc portion of the antibody according to the method of claim 2, the kit comprising:
at least one micropipette tip containing the solid support comprising a protein A affinity medium or a protein G affinity medium;
at least one container containing a labeling solution;
at least one container containing a fluorescent label comprising a reactive group, wherein the fluorescent label is in a form adapted to being dissolved in the labeling solution; and
at least one container containing an elution buffer for eluting fluorescently labeled antibodies from the solid support.

11. The kit according to claim 10, wherein the solid support is fixated inside the solid support container.

12. The kit according to claim 10, wherein the fluorescent label is in freeze dried form.

13. The kit according to claim 10, wherein the eluting buffer has a pH of 4 or lower.

14. The kit according to claim 10, further comprising at least one container containing a washing buffer for washing non-bound material off the solid support.

15. The kit according to claim 10, wherein the reactive group is selected from the group consisting of an amine reactive group and a sulfhydryl reactive group.

16. The kit according to claim 10, wherein the reactive group is an amine reactive group.

17. The kit according to claim 10, wherein the fluorescent label comprises an organic fluorophore.

18. The method according to claim 2, wherein the solid support is a mixture of dextran, polystyrene, and polypropylene.
